(19) United States
(12) Patent Application Publication
(10) Pub. No.: US 2004/0131969 A1
(43) Pub. Date: Jul. 8, 2004

(54) Non-resonant two-photon absorbing material, non-resonant two-photon emitting material, method for inducing absorption of non-resonant two-photons and method for generating emission of non-resonant two-photons

(75) Inventors: Hiroo Takizawa, Kanagawa (JP); Masaharu Akiba, Kanagawa (JP); Takeharu Tani, Kanagawa (JP); Jun Kawamata, Kanagawa (JP); Katsumi Kobayashi, Kanagawa (JP); Karin Kawahara, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/679,446

(22) Filed: Oct. 7, 2003

(30) Foreign Application Priority Data
Oct. 7, 2002 (JP) P. 2002-293809
Jan. 21, 2003 (JP) P. 2003-012417
Mar. 14, 2003 (JP) P. 2003-070303

(51) Int. Cl. G11B 7/24; C07D 263/52; C07D 498/00

(52) U.S. Cl. 430/270.18; 430/945; 548/181; 548/217; 548/238

(57) ABSTRACT

A non-resonant two-photon absorbing material is provided, comprising a specified compound and exhibiting far stronger non-resonant two-photon absorption and two-photon emission than conventional materials.

### FIELD OF THE INVENTION

The present invention relates to a material which reveals a nonlinear optical effect. In particular, the invention relates to an organic nonlinear optical material which has a large non-resonant two-photon absorbing cross sectional area and great luminous efficiency from the excitation state generated by non-resonant two-photon absorption, another object is to provide a non-resonant two-photon absorption-inducing method with the above organic nonlinear optical material, and a further object is to provide non-resonant two-photon emission-generating method.

### BACKGROUND OF THE INVENTION

A nonlinear optical effect generally means nonlinear optical response which is proportioned to the square, cube or more of the applied photoelectric field. As the secondary nonlinear optical effect proportional to the square of the applied photoelectric field, second harmonic generation (SHG), optical rectification, photo-refractive effect, Pockels effect, parametric amplification, parametric oscillation, light summation cycle mixture and light equation cycle mixture are known. As the tertiary nonlinear optical effect proportional to the cube of the applied photoelectric field, third harmonic generation (THG), optical Kerr effect, self-induction refractive index variation and two-photon absorption are exemplified.

A variety of inorganic materials have so far been found as the nonlinear optical materials showing nonlinear optical effect. However, concerning inorganic materials, since so-called molecular design for optimizing desired nonlinear optical characteristics and various physical properties necessary for manufacturing elements is difficult, it has been very difficult to put these inorganic materials to practical use as nonlinear optical materials. On the other hand, with respect to organic materials, desired nonlinear optical characteristics can be optimized by molecular design, and other various physical properties can be controlled as well, therefore, the possibility of the practical use of organic materials is high and they are drawing public attention as promising nonlinear optical materials.

In recent years, of the nonlinear optical characteristics of organic compounds, tertiary nonlinear optical effect is attracting public attention. Above all, non-resonant two-photon absorption and non-resonant two-photon emission are thought to be prospective. Two-photon absorption is a phenomenon which is excited by simultaneous absorption of two photons by a compound, and a case where the absorption of two photons is generated at an energy region where there is not the (linear) absorption band of a compound is called non-resonant two-photon absorption. Non-resonant two-photon emission is emission which is generated by an excited molecule formed by non-resonant two-photon absorption during the course of deactivation of radiation in the excitation state. Two-photon absorption and two-photon emission in the following description indicate non-resonant two-photon absorption and non-resonant two-photon emission respectively unless otherwise indicated.

Non-resonant two-photon absorption efficiency is in proportion to the square of the applied photoelectric field (the square characteristic of two-photon absorption) Accordingly, when a two dimensional plane is irradiated with laser beams, two-photon absorption occurs only at the central part of laser spot where electric field strength is high, and two-photon absorption does not occur at all at the peripheral part where electric field strength is weak. On the other hand, in a three dimensional space, two-photon absorption occurs only at a focal point where laser beams are converged with a lens and electric field strength is high, and two-photon absorption does not occur at all at the verge of the focal point where electric field strength is weak. As compared with linear absorption in which excitation occurs at anywhere in proportion to the strength of the photoelectric field applied, excitation occurs only at one point on the inside of space in non-resonant two-photon absorption due to the square characteristic, so that space resolution is conspicuously improved. In general, in inducing non-resonant two-photon absorption, short pulse laser beams of the near infrared region, where the wavelength is longer than the wavelength region of the (linear) absorption band of a compound and there is not the absorption band of a compound, are used in many cases. Since near infrared rays of the so-called transparent region where there is not the (linear) absorption band of a compound are used, excited light can reach the inside of a sample without being absorbed or scattered. Due to the square characteristic of non-resonant two-photon absorption, it is possible to excite one point of the inside of a sample with markedly high space resolution, therefore, there is every reason to expect that non-resonant two-photon absorption and non-resonant two-photon luminescence can be applied to the fields of, e.g., two-photon shadow-making and photo-dynamic therapy (PDT) of the tissue of living body. Further, since a photon having higher energy than the energy of the photon subjected to incident can be taken out when non-resonant two-photon absorption and non-resonant two-photon emission are used, studies on up-conversion lasing are reported as well from the viewpoint of wavelength conversion device.

So-called stilbazolium derivatives are known as organic compounds which efficiently reveal non-resonant two-photon absorption (refer to He, G. S. et al., Appl. Phys. Lett., Vol. 67, p. 3703 (1995), He, G. S. et al., Appl. Phys. Lett., Vol. 67, p. 2433 (1995), He, G. S. et al., Appl. Phys. Lett., Vol. 68, p. 3549 (1996), He, G. S. et al., J. Appl. Phys., Vol. 81, p. 2529 (1997), Prasad, P. N. et al., Nonlinear Optics, Vol. 21, p. 39 (1999), Ren, Y. et al., J. Mater. Chem., Vol. 10, p. 2025 (2000), Zhou, G. et al., Jpn. J. Appl. Phys., Vol. 40, p. 1250 (2001)), and various application examples using two-photon emission of stilbazolium compounds having certain specific structures are disclosed in patent literature 1. In addition, so-called stilbene derivatives and compounds having certain specific pi-conjugations are also reported as non-resonant two-photon absorbing compounds (Zhou, G. et al., Jpn. J. Appl. Phys., Vol. 40, p. 1250 (2001), Albota, M. et al., Science, Vol. 281, p. 1653 (1998), WO 99/53242 and U.S. Pat. No. 6,267,913).

When non-resonant two-photon emission is applied to shadow-making, photo-dynamic therapy (PDT) of the tissue of living body, micro-shaping, three dimensional optical recording and the like by making use of non-resonant two-photon emission, organic materials used in each of the above applications need to have high two-photon absorption efficiency (a two-photon absorbing cross sectional area). For obtaining molecules in a state of two-photon excitation in double the number with a certain organic compound, four times greater excitation light strength is necessary due to the square characteristic of two-photon absorption. However, irradiation with excessively strong laser beams has high possibilities of damaging the tissue of living body, or of photo-deterioration of two-photon absorbing dyes themselves. Accordingly, for obtaining molecules in a state of two-photon excitation in large numbers with weaker excitation light strength, it is necessary to develop an organic compound capable of effecting two-photon absorption efficiently and a two-photon absorbing material containing the organic compound. The two-photon absorption efficiencies of the compounds and materials described in the above non-patent literature and patent literature are not sufficiently practicable.

In the information-oriented society in recent years, three dimensional optical recording media suddenly came to attract public attention as the ultimate high density and high capacity recording media. Three dimensional optical recording media aim at the achievement of super high density and super high capacity recording several ten to several hundred times as large as conventional two dimensional recording media by repeating bit recording of several ten to several hundred layers in the three dimensional direction (film thickness direction). For providing three dimensional optical recording media, it must be possible to access and write at arbitrary place in the three dimensional direction (film thickness direction), and a method of using a two-photon absorbing material is promising as that means.

In the field of medical treatment, to precisely treat a three dimensionally complicated part, such as a brain or an ear, three dimensional display and three dimensional stereolithography, by which a natural figure in three dimensions can be observed without spectacles by a great number of people, are desired, and three dimensional volume display using two-photon absorption and three dimensional stereolithography composition are expected as promising means for that purpose.

However, for putting three dimensional optical recording medium, three dimensional volume display and stereolithography composition to practical use, high speed recording technique is indispensable. Since a recording speed is in proportion to a two-photon absorbing cross sectional area, when well-known two-photon absorbing compounds having low two-photon absorption efficiency are used, a practical material and a system cannot be proposed, accordingly the development of a compound having an extremely great two-photon absorbing cross sectional area is strongly desired.

Various applications characterized by markedly high space resolution become possible by utilizing non-resonant two-photon absorption and non-resonant two-photon luminescence as described above. However, since two-photon luminescent compounds usable at present time are low in two-photon absorbing power and two-photon luminous efficiency, very high output laser beams are necessary as the excitation light source to induce two-photon absorption and two-photon emission.

### SUMMARY OF THE INVENTION

An object of the present invention is to provide an organic material that efficiently absorbs two photons, i.e., an organic material having a great two-photon absorbing cross sectional area. Another object of the invention is to provide an organic material exhibiting two-photon emission having great luminous intensity. A further object of the invention is to provide a preferable non-resonant two-photon absorption-inducing method and non-resonant two-photon emission-generating method with the organic material.

1. A non-resonant two-photon absorbing material comprising a compound represented by the following formula (1) or (3):

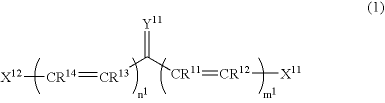

wherein R¹¹, R¹², R¹³ and R¹⁴ each independently represents a hydrogen atom or a substituent, and some of R¹¹, R¹², R¹³ and R¹⁴ may be bonded to each other to form a ring; m¹ and n¹ each represents an integer of from 0 to 4, and when m¹ and n¹ each represents 2 or more, a plurality of R¹¹, R¹², R¹³ and R¹⁴ may be the same or different; X¹¹ and X¹², which may be the same or different, each represents a substituted or unsubstituted aryl group, or a substituted or unsubstituted heterocyclic group; Y¹¹ represents an atomic group containing at least one of partial structures represented by the following formulae (2-1) to (2-6):

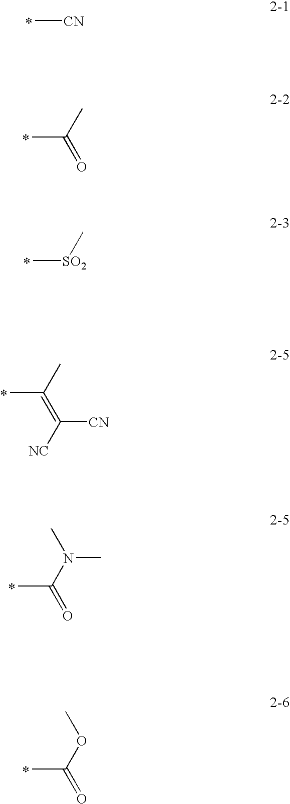

wherein * represents the bonding position in Y¹¹, when Y¹¹ contains two of partial structures represented by formulae (2-1) to (2-6), the two partial structures may be different from each other, and may be bonded to form a ring, and when Y¹¹ contains one of partial structure represented by formulae (2-1) to (2-6), other necessary atom or atomic group may be arbitrary;

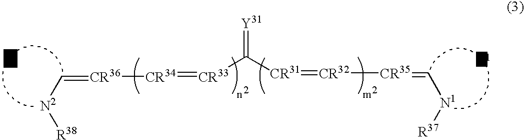

wherein R³¹, R³², R³³, R³⁴, R³⁵ and R³⁶ each independently represents a hydrogen atom or a substituent, and some of R³¹, R³², R³³, R³⁴, R³⁵ and R³⁶ may be bonded to each other to form a ring; m² and n² each represents an integer of from 1 to 4, and when m² and n² each represents 2 or more, a plurality of R³¹, R³², R³³ and R³⁴ may be the same or different; R³⁷ and R³⁸ each represents a hydrogen atom, an alkyl group, an alkenyl group, an aryl group, or a heterocyclic group; Z¹ and Z² each represents an atomic group to form a 5- or 6-membered ring; Y³¹ represents an oxygen atom, or an atomic group containing at least one of partial structures represented by the following formulas (2-1) to (2-6):

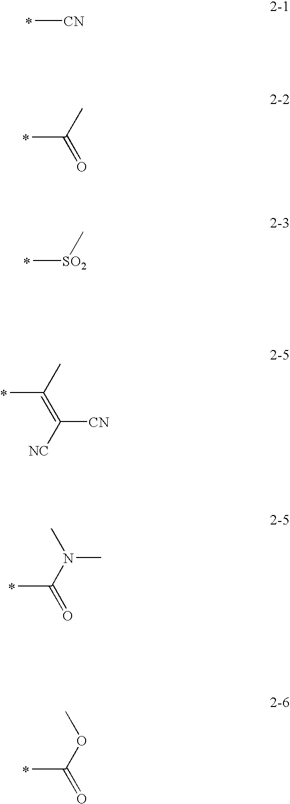

wherein * represents the bonding position in Y³¹, when Y³¹ contains two of partial structures represented by formulae (2-1) to (2-6), the two partial structures may be different from each other, and may be bonded to form a ring, and when Y³¹ contains one of partial structure represented by formulae (2-1) to (2-6), other necessary atom or atomic group may be arbitrary.

2. The non-resonant two-photon absorbing material as described in the item 1, wherein Y¹¹ contains two partial structures represented by the formula (2-1).

3. The non-resonant two-photon absorbing material as described in the item 1 or 2, wherein Y³¹ contains two partial structures represented by the formula (2-1).

4. The non-resonant two-photon absorbing material as described in the item 1 or 2, wherein Y³¹ represents an oxygen atom.

5. The non-resonant two-photon absorbing material as described in any one of the items 1 to 4, wherein the ring formed by Z¹ and Z² is an indolenine ring or an azaindolenine ring.

6. The non-resonant two-photon absorbing material as described in any one of the items 1 to 5, wherein R¹¹ and R¹³, or R³¹ and R³³ are bonded to each other to form a ring.

7. The non-resonant two-photon absorbing material as described in any one of the items 1 to 6, wherein X¹¹ and X¹² each represents a phenyl group, a naphthyl group or a nitrogen-containing heterocyclic group.

8. The non-resonant two-photon absorbing material as described in any one of the items 1 to 7, wherein the compound represented by the formula (1) or (3) has a hydrogen bonding group in the molecule.

9. The non-resonant two-photon absorbing material as described in the item 8, wherein the hydrogen bonding group is selected

from —COOH, —CONHR¹, —SO₃H, —SO₂NHR², —P(O) (OH)OR³, —OH, —SH, —NHR⁴, —NHCOR⁵ and —NR⁶C(O)NHR⁷, in which R¹ and R² each independently represents a hydrogen atom, an alkyl group, an alkenyl group, an aryl group, a heterocyclic group, —COR⁸ or —SO₂R⁹; R³ to R⁹ each indenpendently represents a hydrogen atom, an alkyl group, an alkenyl group, an aryl group or a heterocyclic group.

10. The non-resonant two-photon absorbing material as described in any one of the items 1 to 9, wherein the compound represented by formula (1) or (3) has a two-photon absorbing cross sectional area of 1,000 GM or more.

11. A non-resonant two-photon emitting material comprising the compound represented by formula (1) or (3) as described in any one of the items 1 to 10.

12. A method for inducing a non-resonant two-photon absorption, which comprises irradiating the compound represented by formula (1) or (3) as described in any one of the items 1 to 10 with laser beams having a wavelength longer than the linear absorption band of the compound to induce a two-photon absorption.

13. A method for generating an emission of non-resonant two-photon, which comprises irradiating the compound represented by formula (1) or (3) as described in any one of the items 1 to 10 with laser beams having a wavelength longer than the linear absorption band of the compound to induce non-resonant two-photon absorption and generate an emission.

14. An optical recording medium comprising the non-resonant two-photon absorbing material as described in any one of the items 1 to 10.

15. A three dimensional optical recording medium comprising the non-resonant two-photon absorbing material as described in any one of the items 1 to 10.

16. A three dimensional volume display comprising the non-resonant two-photon absorbing material as described in any one of the items 1 to 10.

17. A three dimensional stereolithography material comprising the non-resonant two-photon absorbing material as described in any one of the items 1 to 10.

### DETAILED DESCRIPTION OF THE INVENTION

The compounds according to the present invention represented by formula (1) or (3) are described in detail below. In the present invention, when a specific part is called “a group”, it means that the group may be substituted with one or more substituents (until the possible maximum) or may not be substituted. For instance, “an alkyl group” means “a substituted or unsubstituted alkyl group”. The substituents usable in the compounds of the present invention are restricted by no means and every substituent can be used.

Further, in the present invention, when a specific part is called “a ring”, or “a ring” is contained in “a group”, the ring may be a monocyclic ring or a condensed ring, and may be substituted or unsubstituted, unless otherwise indicated.

For instance, “an aryl group” may be a phenyl group, a naphthyl group, or a substituted phenyl group.

In formula (1), R¹¹, R¹², R¹³ and R¹⁴ each represents a hydrogen atom or a substituent. The preferred examples of the substituents include an alkyl group (preferably an alkyl group having from 1 to 20 carbon atoms, e.g., methyl, ethyl, n-propyl, isopropyl, n-butyl, n-pentyl, benzyl, 3-sulfopropyl, 4-sulfobutyl, carboxymethyl and 5-carboxypentyl), an alkenyl group (preferably an alkenyl group having from 2 to 20 carbon atoms, e.g., vinyl and allyl), a cycloalkyl group (preferably a cycloalkyl group having from 3 to 20 carbon atoms, e.g., cyclopentyl and cyclohexyl), an aryl group (preferably an aryl group having from 6 to 20 carbon atoms, e.g., phenyl, 2-chlorophenyl, 4-methoxyphenyl, 3-methylphenyl and 1-naphthyl), an alkoxyl group (preferably an alkoxyl group having from 1 to 16 carbon atoms, e.g., methoxy, ethoxy, butoxy and cyclohexyloxy), an aryloxy group (preferably an aryloxy group having from 6 to 14 carbon atoms, e.g., phenoxy and 1-naphthoxy), an amino group (an amino group having from 0 to 20 carbon atoms, e.g., dimethylamino, diethylamino and dibutylamino), a halogen atom, an alkoxycarbonyl group (an alkoxycarbonyl group having from 2 to 17 carbon atoms, e.g., methoxycarbonyl, ethoxycarbonyl and t-butylcarbonyl), a carbamoyl group (a carbamoyl group having from 1 to 10 carbon atoms, e.g., carbamoyl, N-methylcarbamoyl and N,N-dimethylcarbamoyl), an acylamino group (an acylamino group having from 1 to 10 carbon atoms, e.g., formylamino), and a heterocyclic group (preferably a heterocyclic group having from 1 to 20 carbon atoms, e.g., pyridyl, thienyl, furyl, thiazolyl, imidazolyl, pyrazolyl, pyrrolidino, piperidino and morpholino).

R¹¹, R¹², R¹³ and R¹⁴ each more preferably represents a hydrogen atom or an alkyl group. Some of R¹¹, R¹², R¹³ and R¹⁴ may be bonded to each other to form a ring (e.g., a 4- to 7-membered ring), and when a ring is formed, the preferred examples of the rings include a cyclopentane ring, a cyclobutane ring, a cyclohexane ring, a cyclohexene ring, a cyclopentene ring and a cycloheptadienone ring.

In formula (1), m¹ and n¹ each represents an integer of from 0 to 4, and preferably an integer of from 2 to 4. When m¹ and n¹ each represents 2 or more, a plurality of R¹¹, R¹², R¹³ and R¹⁴ may be the same or different.

In formula (1), X¹¹ and X¹² each represents a substituted or unsubstituted aryl group, or a substituted or unsubstituted heterocyclic group. The examples of the aryl groups represented by X¹¹ and X¹² include phenyl, naphthylanthracenyl and phenanthrenyl, preferably phenyl and naphthyl, and particularly preferably phenyl.

In formula (1), the heterocyclic group represented by X¹¹ and X¹² is a heterocyclic group having from 1 to 15 carbon atoms, and more preferably a heterocyclic group having from 2 to 12 carbon atoms. A nitrogen atom, an oxygen atom and a sulfur atom are preferred as the hetero atoms.

The specific examples of the heterocyclic rings include, e.g., pyrrolidine, piperidine, piperazine, morpholine, thiophene, selenophene, furan, pyrrole, imidazole, pyrazole, pyridine, pyrazine, pyridazine, pyrimidine, triazole, triazine, indole, indazole, purine, thiazoline, thiazole, thiadiazole, oxazoline, oxazole, oxadiazole, quinoline, isoquinoline, phthalazine, naphthyridine, quinoxaline, quinazoline, cinnoline, butylidyne, acridine, phenanthroline, phenazine, tetrazole, benzimidazole, benzoxazole, benzothiazole, benzotriazole, tetraazaindene, benzindolenine, carbazole, dibenzofuran, phenothiazine, julolidine, and in a case where nitrogen atoms form a ring, the quaternary onium cations of the quaternized nitrogen atoms. The preferred examples of the heterocyclic rings are pyridine, pyrimidine, pyrazine, indole, thiophene, thiazole, oxazole, quinoline, acridine, benzimidazole, benzoxazole, benzothiazole, benzindolenine, carbazole, phenothiazine, julolidine, and in the case where nitrogen atoms form a ring, quaternary onium cations of the quaternized nitrogen atoms, and more preferred heterocyclic rings are carbazole, phenothiazine and julolidine.

X¹¹ and X¹² in formula (1) may further be substituted, and as the examples of the preferred substituents, the same substituents as described above as the substituents of the groups represented by R¹¹, R¹², R¹³ and R¹⁴ can be exemplified.

Y¹¹ represents an atomic group containing at least one of the partial structures represented by the following formulae (2-1) to (2-6).

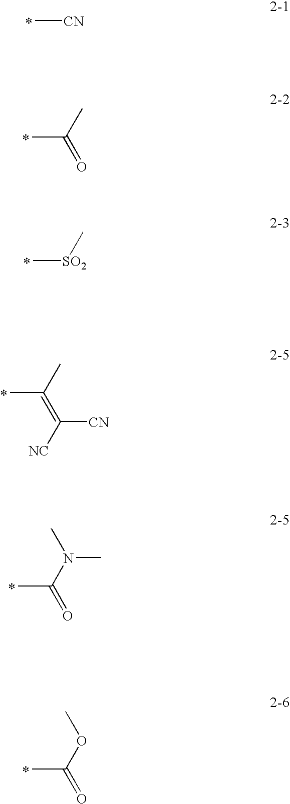

In each compound represented by formulae (2-1) to (2-6), * represents the bonding position in Y¹¹, when Y¹¹ contains two of partial structures represented by formulae (2-1) to (2-6), they may be different from each other, and they may be bonded to form a ring, and when Y¹¹ contains one of partial structures represented by formulae (2-1) to (2-6), other necessary atom or atomic group may be arbitrary. The case where Y¹¹ contains two partial structures represented by formulae (2-1) to (2-6) is preferred.

The examples of the atomic groups represented by Y¹¹ containing partial structures represented by the formulae (2-1) to (2-6) include the structures represented by the following formula (5).

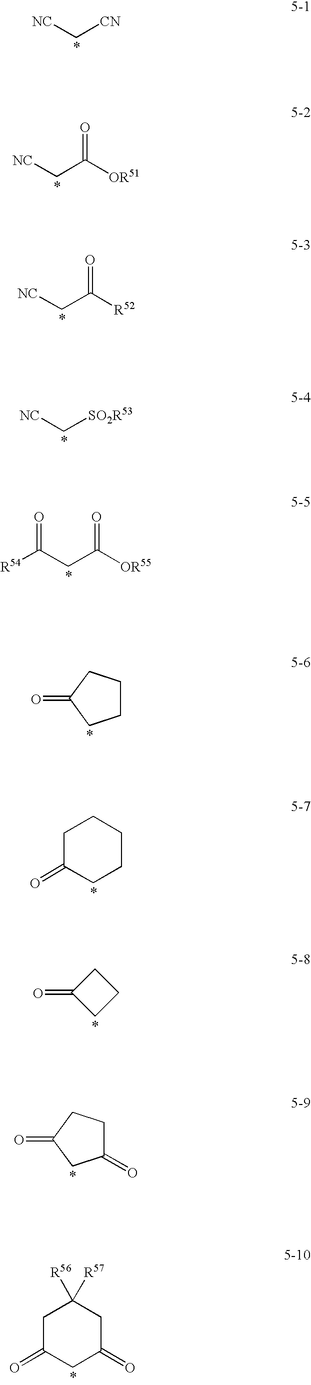

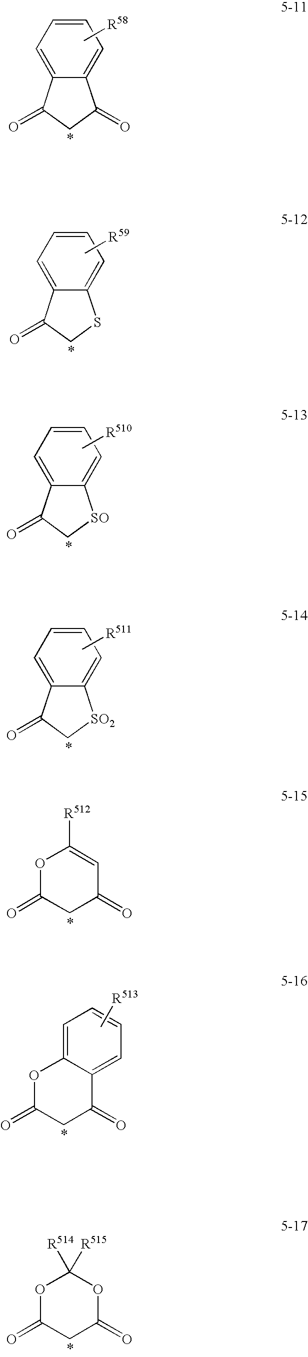

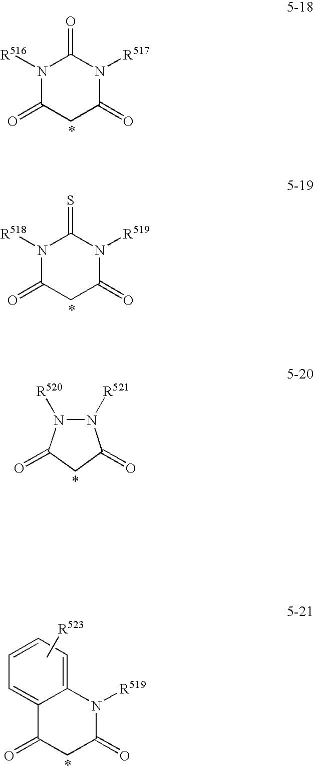

In formula (5), R⁵¹, R⁵², R⁵³, R⁵⁴, R⁵⁵, R⁵⁶, R⁵⁷, R⁵⁸, R⁵⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, R⁵²² and R⁵²³ each represents a hydrogen atom or a substituent, and the examples of the substituents are the same as those described above in the substituents of the groups represented by R¹¹, R¹², R¹³ and R¹⁴. The preferred examples of the substituents represented by R⁵¹, R⁵², R⁵³, R⁵⁴, R⁵⁵, R⁵⁶, R⁵⁷, R⁵⁸, R⁵⁹, R⁵¹⁰, R⁵¹¹, R⁵¹², R⁵¹³, R⁵¹⁴, R⁵¹⁵, R⁵¹⁶, R⁵¹⁷, R⁵¹⁸, R⁵¹⁹, R⁵²⁰, R⁵²¹, R⁵²² and R⁵²³ in formula (5) include a substituted or unsubstituted alkyl group (more preferably a substituted or unsubstituted having from 1 to 20 carbon atoms, e.g., methyl, ethyl, n-propyl, isopropyl, n-butyl, n-pentyl, benzyl, 3-sulfopropyl, 4-sulfobutyl, carboxymethyl and 5-carboxypentyl), a substituted or unsubstituted cycloalkyl group (more preferably a substituted or unsubstituted cycloalkyl group having from 3 to 20 carbon atoms, e.g., cyclopentyl and cyclohexyl), and a substituted or unsubstituted aryl group (more preferably a substituted or unsubstituted aryl group having from 6 to 20 carbon atoms, e.g., phenyl, 2-chlorophenyl, 4-methoxyphenyl, 3-methylphenyl and 1-naphthyl). When R⁵⁶ and R⁵⁷, and R⁵¹⁴ and R⁵¹⁵ in formula (5) represent alkyl groups, they may be bonded to each other to form a ring.

Y¹¹ in formula (1) is preferably the structure containing any two atomic groups represented by formula (2), more preferably the atomic group represented by (5-1), (5-17), (5-18) or (5-19) of formula (5), still more preferably the atomic group represented by (5-1), (5-18) or (5-19), and most preferably (5-1).

In formula (3), R³¹, R³², R³³, R³⁴, R³⁵ and R³⁶ each represents a hydrogen atom or a substituent. The preferred examples of the substituents include an alkyl group (preferably an alkyl group having from 1 to 20 carbon atoms, e.g., methyl, ethyl, n-propyl, isopropyl, n-butyl, n-pentyl, benzyl, 3-sulfopropyl, 4-sulfobutyl, carboxymethyl and 5-carboxypentyl), an alkenyl group (preferably an alkenyl group having from 2 to 20 carbon atoms, e.g., vinyl and allyl), a cycloalkyl group (preferably a cycloalkyl group having from 3 to 20 carbon atoms, e.g., cyclopentyl and cyclohexyl), an aryl group (preferably an aryl group having from 6 to 20 carbon atoms, e.g., phenyl, 2-chlorophenyl, 4-methoxyphenyl, 3-methylphenyl and 1-naphthyl), an alkoxyl group (preferably an alkoxyl group having from 1 to 16 carbon atoms, e.g., methoxy, ethoxy, butoxy and cyclohexyloxy), an aryloxy group (preferably an aryloxy group having from 6 to 14 carbon atoms, e.g., phenoxy and 1-naphthoxy), an amino group (an amino group having from 0 to 20 carbon atoms, e.g., dimethylamino, diethylamino and dibutylamino), a halogen atom, an alkoxycarbonyl group (an alkoxycarbonyl group having from 2 to 17 carbon atoms, e.g., methoxycarbonyl, ethoxycarbonyl and t-butylcarbonyl), a carbamoyl group (a carbamoyl group having from 1 to 10 carbon atoms, e.g., carbamoyl, N-methylcarbamoyl and N,N-dimethylcarbamoyl), an acylamino group (an acylamino group having from 1 to 10 carbon atoms, e.g., formylamino), and a heterocyclic group (preferably a heterocyclic group having from 1 to 20 carbon atoms, e.g., pyridyl, thienyl, furyl, thiazolyl, imidazolyl, pyrazolyl, pyrrolidino, piperidino and morpholino).

R³¹, R³², R³³, R³⁴, R³⁵ and R³⁶ each preferably represents a hydrogen atom or an alkyl group.

Some of R³¹, R³², R³³, R³⁴, R³⁵ and R³⁶ may be bonded to each other to form a ring (e.g., a 4- to 7-membered ring). When rings are formed, the preferred examples of the rings include a cyclopentane ring, a cyclobutane ring, a cyclohexane ring, a cyclohexene ring, a cyclopentene ring and a cycloheptadienone ring.

In formula (3), m² and n² each represents an integer of from 1 to 4, and preferably an integer of from 1 to 3. When m² and n² each represents 2 or more, a plurality of R³¹, R³², R³³ and R³⁴ may be the same or different.

In formula (3), R³⁷ and R³⁸ each represents a hydrogen atom, an alkyl group, an alkenyl group, an aryl group, or a heterocyclic group, (the preferred examples of the substituents are the same as those in R³¹ to R³⁶), preferably an alkyl group, more preferably an unsubstituted alkyl group, or an alkyl group substituted with a sulfo group or a carboxyl group, and still more preferably an unsubstituted alkyl group having from 1 to 6 carbon atoms or a sulfoalkyl group having from 1 to 4 carbon atoms.

In formula (3), Z¹ and Z² each represents an atomic group to form a 5- or 6-membered ring. The examples of the heterocyclic rings formed include an indolenine ring, an azaindolenine ring, a pyrazoline ring, a benzothiazole ring, a thiazole ring, a thiazoline ring, a benzoxazole ring, an oxazole ring, an oxazoline ring, a benzimidazole ring, an imidazole ring, a thiadiazole ring, a quinoline ring and a pyridine ring, more preferably an indolenine ring, an azaindolenine ring, a pyrazoline ring, a benzothiazole ring, a thiazole ring, a thiazoline ring, a thiadiazole ring and a quinoline ring, and particularly preferably an indolenine ring and an azaindolenine ring.

In formula (3), the heterocyclic ring formed by Z¹ and Z² may have a substituent, and the examples of the preferred substituents include an alkyl group (preferably an alkyl group having from 1 to 20 carbon atoms, e.g., methyl, ethyl, n-propyl, isopropyl, n-butyl, n-pentyl, benzyl, 3-sulfopropyl, 4-sulfobutyl, carboxymethyl and 5-carboxypentyl), an alkenyl group (preferably an alkenyl group having from 2 to 20 carbon atoms, e.g., vinyl and allyl), a cycloalkyl group (preferably a cycloalkyl group having from 3 to 20 carbon atoms, e.g., cyclopentyl and cyclohexyl), an aryl group (preferably an aryl group having from 6 to 20 carbon atoms, e.g., phenyl, 2-chlorophenyl, 4-methoxyphenyl, 3-methylphenyl and 1-naphthyl), a heterocyclic group (preferably a heterocyclic group having from 1 to 20 carbon atoms, e.g., pyridyl, thienyl, furyl, thiazolyl, imidazolyl, pyrazolyl, pyrrolidino, piperidino and morpholino), an alkynyl group (preferably an alkynyl group having from 2 to 20 carbon atoms, e.g., ethynyl, 2-methylethynyl and 2-phenylethynyl), a halogen atom (e.g., F, Cl, Br and I), an amino group (preferably an amino group having from 1 to 20 carbon atoms, e.g., dimethylamino, diethylamino and dibutylamino), a cyano group, a hydroxyl group, a carboxyl group, a sulfo group, an acyl group (preferably an acyl group having from 1 to 20 carbon atoms, e.g., acetyl, benzoyl, salicyloyl and pivaloyl), an alkoxyl group (preferably an alkoxyl group having from 1 to 20 carbon atoms, e.g., methoxy, butoxy and cyclohexyloxy), an aryloxy group (preferably an aryloxy group having from 6 to 26 carbon atoms, e.g., phenoxy and 1-naphthoxy), an alkylthio group (preferably an alkylthio group having from 1 to 20 carbon atoms, e.g., methylthio and ethylthio), an arylthio group (preferably an arylthio group having from 6 to 20 carbon atoms, e.g., phenylthio and 4-chlorophenylthio), an alkylsulfonyl group (preferably an alkylsulfonyl group having from 1 to 20 carbon atoms, e.g., methanesulfonyl and butanesulfonyl), an arylsulfonyl group (preferably an arylsulfonyl group having from 6 to 20 carbon atoms, e.g., benzenesulfonyl and paratoluenesulfonyl), a carbamoyl group (preferably a carbamoyl group having from 1 to 20 carbon atoms, e.g., N,N-dimethylcarbamoyl and N-phethylcarbamoyl), an acylamino group (preferably an acylamino group having from 1 to 20 carbon atoms, e.g., acetylamino and benzoylamino), an imino group (preferably an imino group having from 2 to 20 carbon atoms, e.g., phthalimino), an acyloxy group (preferably an acyloxy group having from 1 to 20 carbon atoms, e.g., acetyloxy and benzoyloxy), and an alkoxycarbonyl group (preferably an alkoxycarbonyl group having from 2 to 20 carbon atoms, e.g., methoxycarbonyl and phenoxycarbonyl), and more preferably an alkyl group, an aryl group, a heterocyclic group, a halogen atom, a carboxyl group (including the salts of a carboxyl group as well), a sulfo group (including the salts of a sulfo group as well), an alkoxyl group, a carbamoyl group, and an alkoxycarbonyl group.

Y³¹ represents an oxygen atom, or an atomic group containing at least one of the partial structures represented by formulae (2). In formula (2), * represents the bonding position in Y³¹. When Y³¹ contains two atomic groups represented by formula (2), they may be different from each other, and they maybe bonded to form a ring. When Y³¹ contains one atomic group represented by formula (2), other necessary atom or atomic group may be arbitrary. The case where Y³¹ contains two atomic groups represented by formula (2) is preferred.

As the examples of the atomic groups represented by Y³¹ containing partial structures represented by (2-1) to (2-6) of formula (2), the structures represented by formula (5) are exemplified. The definitions and the preferred embodiments in formula (3) are also the same as those in formula (1).

Y³¹ in formula (3) preferably represents an oxygen atom, or the structure containing any two atomic groups represented by formula (2), and in the latter case, more preferably the atomic group represented by (5-1), (5-17), (5-18) or (5-19) of formula (5), still more preferably the atomic group represented by (5-1), (5-18) or (5-19), and most preferably (5-1).

It is preferred for the compound represented by formula (1) or (3) to have a hydrogen bonding group in the molecule. The hydrogen bonding group in the present invention means a hydrogen-donating group or a hydrogen-accepting group in hydrogen bonding, and a group having both properties is more preferred.

It is preferred that the compound having a hydrogen bonding group undergo aggregation interaction by the interaction of hydrogen bonding groups with each other in a solution state or a solid state. The interaction may be either intramolecular or intermolecular interaction but intermolecular interaction is more preferred.

The hydrogen bonding group is preferably selected from —COOH, —CONHR¹, —SO₃H, —SO₂NHR², —P(O) (OH)OR³, —OH, —SH, —NHR⁴, —NHCOR⁵, and —NR⁶C(O)NHR⁷, wherein R¹ and R² each represents a hydrogen atom, an alkyl group (preferably a alkyl group having from 1 to 20 carbon atoms, e.g., methyl, ethyl, n-propyl, isopropyl, n-butyl, n-pentyl, benzyl, 3-sulfopropyl, 4-sulfobutyl, carboxymethyl and 5-carboxypentyl), an alkenyl group (preferably an alkenyl group having from 2 to 20 carbon atoms, e.g., vinyl and allyl), an aryl group (preferably an aryl group having from 6 to 20 carbon atoms, e.g., phenyl, 2-chlorophenyl, 4-methoxyphenyl, 3-methylphenyl and 1-naphthyl), a heterocyclic group (preferably a heterocyclic group having from 1 to 20 carbon atoms, e.g., pyridyl, thienyl, furyl, thiazolyl, imidazolyl, pyrazolyl, pyrrolidino, piperidino and morpholino), a —COR⁸ group or an —SO₂R⁹ group, wherein R³ to R⁹ each represents a hydrogen atom, an alkyl group, an alkenyl group, an aryl group or a heterocyclic group (the preferred examples are the same as those in R¹ and R²).

R¹ preferably represents a hydrogen atom, an alkyl group, an aryl group, a —COR⁸ group or an —SO₂R⁹ group, wherein R⁸ and R⁹ each preferably represents an alkyl group or an aryl group.

R¹ more preferably represents a hydrogen atom, an alkyl group or an —SO₂R⁹ group, and most preferably a hydrogen atom.

R² preferably represents a hydrogen atom, an alkyl group, an aryl group, a —COR⁸ group or an —SO₂R⁹ group, wherein R⁸ and R⁹ each preferably represents an alkyl group or an aryl group.

R² more preferably represents a hydrogen atom, an alkyl group or a —COR⁸ group, and most preferably a hydrogen atom.

R³ preferably represents a hydrogen atom, an alkyl group or an aryl group, and more preferably a hydrogen atom.

R⁴ preferably represents a hydrogen atom, an alkyl group or an aryl group.

R⁵ preferably represents an alkyl group or an aryl group.

R⁶ preferably represents a hydrogen atom, and R⁷ preferably represents a hydrogen atom, an alkyl group or an aryl group.

The hydrogen bonding group is more preferably any one selected from —COOH, —CONHR¹, —SO₂NHR², —NHCOR⁵, and —NR⁶C(O)NHR⁷, still more preferably any of —COOH, —CONHR¹, and —SO₂NHR², and most preferably either —COOH or —CONH₂.

When the compound represented by formula (1) or (3) has a hydrogen bonding group, the hydrogen bonding group maybe contained anywhere, but it is preferred that the hydrogen bonding group be contained on the ring formed by Z¹ and Z² in formula (3), or contained in X¹¹ and X¹² in formula (1).

The specific examples of the preferred hydrogen bonding groups contained in the non-resonant two-photon absorbing compounds having a hydrogen bonding group in the molecule which reveal non-resonant two-photon absorption are shown below, but the present invention is not limited to these compounds.

| | |
|---|---|
| H-1 | —COOH |
| H-2 | —CONH₂ |
| H-3 | —CONHCH₃ |
| H-4 | 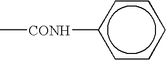 |
| H-5 | —CONHCH₂CH═CH₂ |
| H-6 | 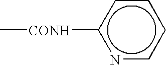 |
| H-7 | —CONHCOCH₃ |
| H-8 | —CONHSO₂CH₃ |
| H-9 | —SO₂NH₂ |
| H-10 | —SO₂NHCH₃ |
| H-11 | 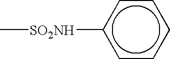 |
| H-12 | —SO₂NHSO₂CF₃ |
| H-13 | —SO₂NHCOCH₃ |
| H-14 | —SO₃H |
| H-15 | 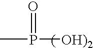 |
| H-16 | 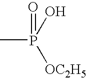 |
| H-17 | 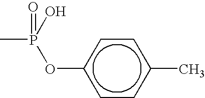 |
| H-18 | —OH |
| H-19 | —SH |
| H-20 | —NHCH₃ |
| H-21 | 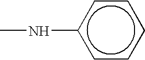 |
| H-22 | —NHCOCH₃ |
| H-23 | 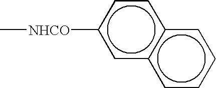 |
| H-24 | —NHCONH₂ |
| H-25 | —NHCONHCH₃ |
| H-20 | 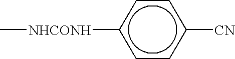 |

The specific examples of the preferred two-photon absorbing compounds and two-photon luminescent compounds represented by formula (1) or (3) which are used in the present invention are shown below, but the present invention is not limited thereto.

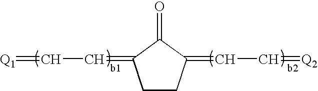

| | Q₁ | Q₂ | b₁ | b₂ |
|---|---|---|---|---|
| D-1 | 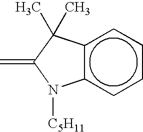 | 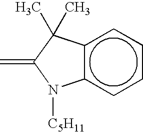 | 2 | 2 |
| D-2 | ″ | ″ | 1 | 1 |
| D-3 | ″ | ″ | 3 | 3 |
| D-4 | 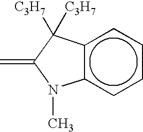 | 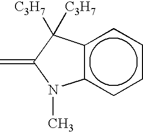 | 2 | 3 |
| D-5 | 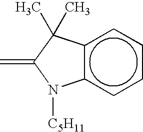 | 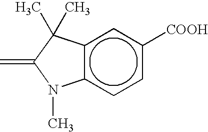 | 2 | 2 |
| D-6 | 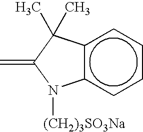 | 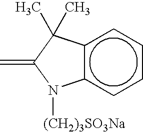 | 2 | 2 |
| D-7 | 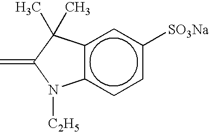 | 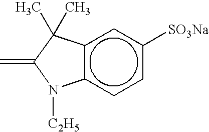 | 2 | 2 |
| D-8 | 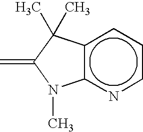 | 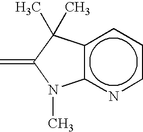 | 2 | 2 |
| D-9 | 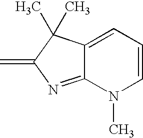 | 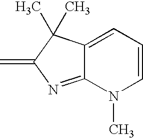 | 2 | 2 |
| D-10 | 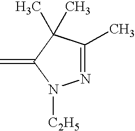 | 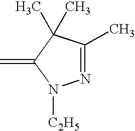 | 2 | 2 |
| D-11 | 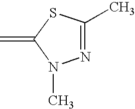 | 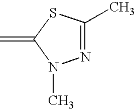 | 1 | 1 |
| D-12 | 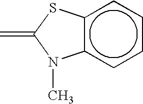 | 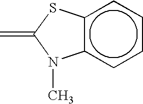 | 1 | 1 |
| D-13 | 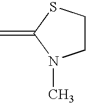 | 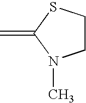 | 1 | 1 |
| D-14 | 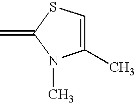 | 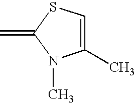 | 1 | 1 |
| D-15 | 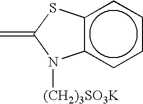 | 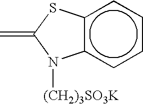 | 1 | 1 |
| D-16 | 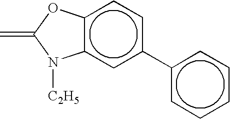 | 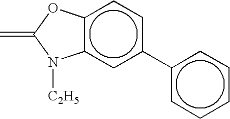 | 1 | 1 |
| D-17 | 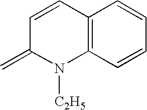 | 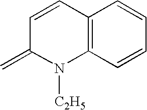 | 1 | 1 |
| D-18 | 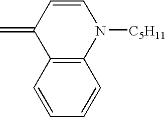 | 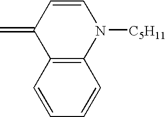 | 1 | 1 |

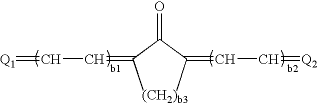

| | Q₁ | Q₂ | b₁ | b₂ | b₃ |
|---|---|---|---|---|---|
| D-19 | 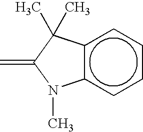 | 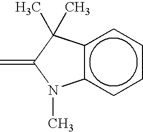 | 2 | 2 | 1 |
| D-20 | ″ | ″ | ″ | ″ | 3 |
| D-21 | ″ | ″ | ″ | ″ | 4 |
| D-22 | 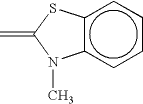 | 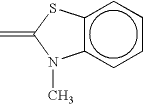 | 1 | 1 | 3 |
| D-23 | 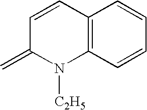 | 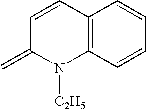 | ″ | ″ | ″ |
| D-24 | 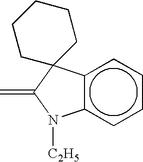 | 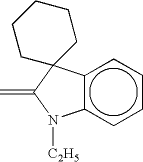 | 2 | 2 | 2 |
| D-25 | 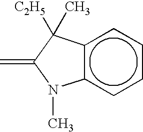 | 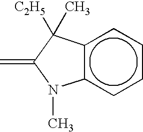 | 2 | 2 | ″ |
| D-26 | 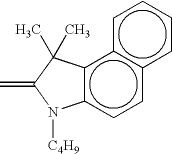 | 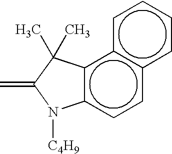 | 2 | 2 | ″ |
| D-27 | ″ | ″ | 1 | 1 | ″ |

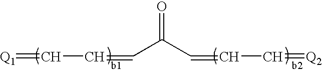

| | Q₁ | Q₂ | b₁ | b₂ |
|---|---|---|---|---|
| D-28 | 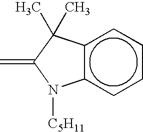 | 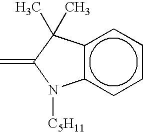 | 1 | 1 |
| D-29 | ″ | ″ | 2 | 2 |
| D-30 | ″ | ″ | 3 | 3 |

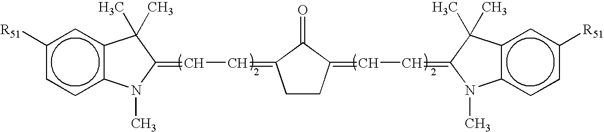

| | R₅₁ |
|---|---|
| D-31 | —Cl |
| D-32 | —OCH₃ |
| D-33 | —CONHCH₃ |
| D-34 | —CN |
| D-35 | —COOH |
| D-36 | 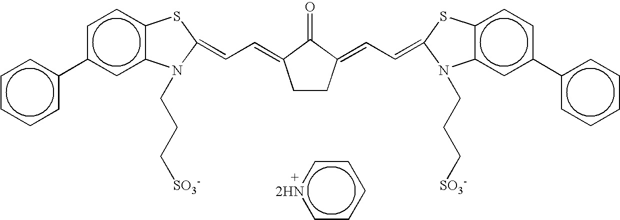 |
| D-37 | 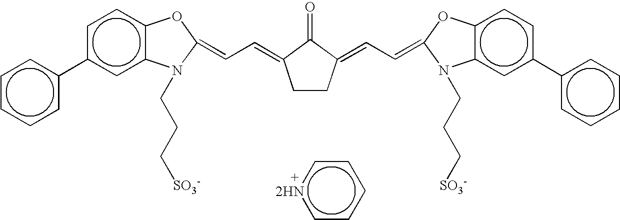 |

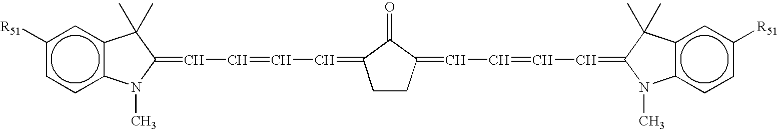

| | R₃₁ |
|---|---|
| DD-1 | —COOH |
| DD-2 | —CONH₂ |
| DD-3 | —CONHCH₃ |
| DD-4 | 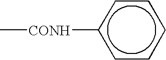 |
| DD-5 | —CONHCOCH₃ |
| DD-6 | —CONHSO₂CH₃ |
| DD-7 | —SO₂NH₂ |
| DD-8 | —SO₂NHCH₃ |
| DD-9 | 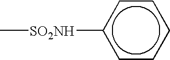 |
| DD-10 | —SO₃H |
| DD-11 | 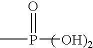 |
| DD-12 | —OH |
| DD-13 | —SH |
| DD-14 | —NHCH₃ |
| DD-15 | —NHCOCH₃ |
| DD-16 | —NHCONH₂ |

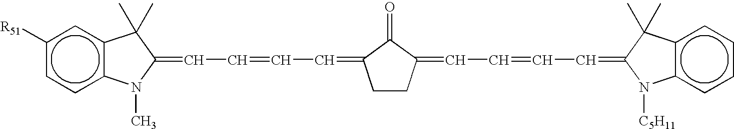

| | R₅₁ |
|---|---|
| DD-17 | —COOH |
| DD-18 | —CONH₂ |
| DD-19 | —SO₂NH₂ |
| DD-20 | 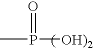 |

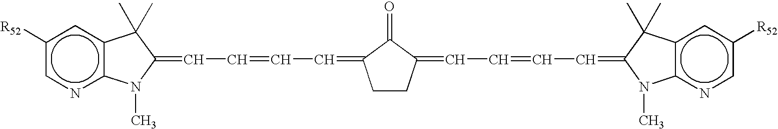

| | R₅₂ |
|---|---|
| DD-21 | —COOH |
| DD-22 | —CONH₂ |
| DD-23 | —SO₂NH₂ |
| DD-24 | 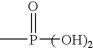 |

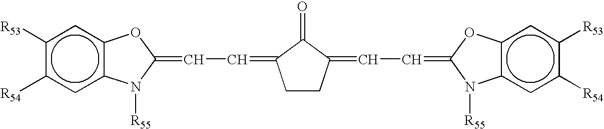

| | R₅₃ | R₅₄ | R₅₅ |
|---|---|---|---|
| DD-25 | —COOH | —H | 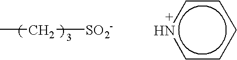 |
| DD-26 | —CONH₂ | ″ | ″ |
| DD-27 | ″ | ″ | —C₄H₉ |
| DD-28 | H | —SO₂NH₂ | 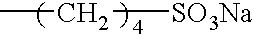 |
| DD-29 | ″ | —SO₃H | —C₂H₅ |
| DD-30 | ″ | 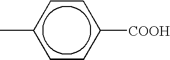 | 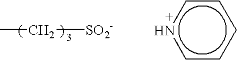 |

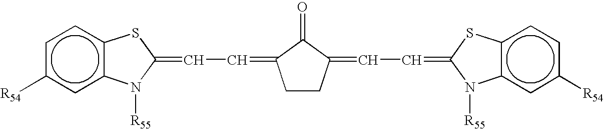

| | R₅₄ | R₅₅ |
|---|---|---|
| DD-31 | —COOH | 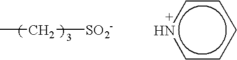 |
| DD-32 | ″ | —CH₃ |
| DD-33 | —CONH₂ | 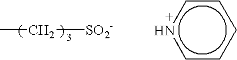 |
| DD-34 | —SO₂NH₂ | ″ |

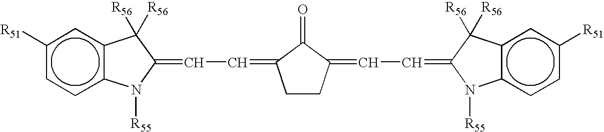

| | R₅₁ | R₅₅ | R₅₆ |
|---|---|---|---|
| DD-35 | —COOH | 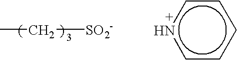 | —CH₃ |
| DD-36 | ″ | —CH₃ | —C₃H₇ |
| DD-37 | —SO₃H | —CH₂COOH | —CH₃ |
| DD-38 | —CONH₂ | 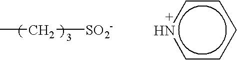 | ″ |
| DD-39 | ″ | —C₂H₅ | ″ |
| DD-40 | —SO₂NH₂ | 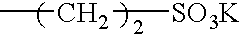 | ″ |
| DD-41 | —H | —CH₂COOH | ″ |

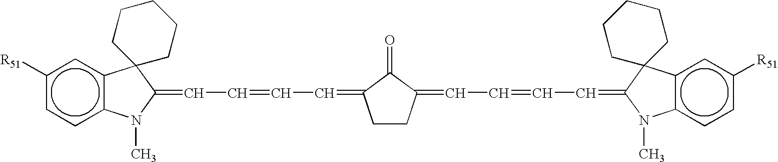

| | R₅₁ |
|---|---|
| DD-42 | —COOH |
| DD-43 | —CONH₂ |

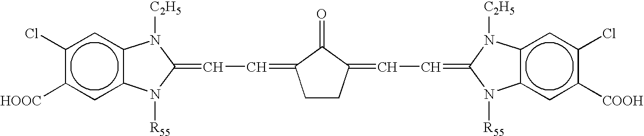

| | R₅₅ |
|---|---|
| DD-44 | 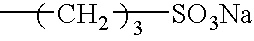 |
| DD-45 | —C₂H₅ |

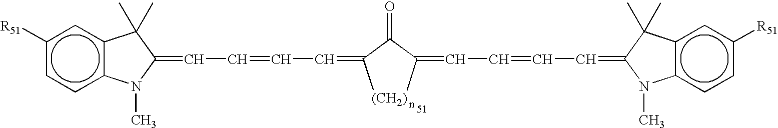

| | R₅₁ | n₅₁ |
|---|---|---|
| DD-46 | —COOH | 1 |
| DD-47 | ″ | 3 |
| DD-48 | ″ | 4 |
| DD-49 | —CONH₂ | 1 |
| DD-50 | ″ | 3 |

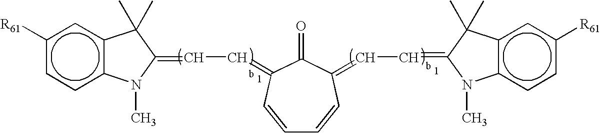

| | b₁ | R₆₁ |
|---|---|---|
| DD-51 | 1 | —H |
| DD-52 | 1 | —CONH₂ |
| DD-53 | 2 | —H |

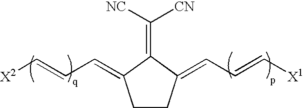

| | X¹ | X² | p | q |
|---|---|---|---|---|
| A-1 | 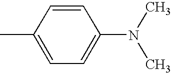 | 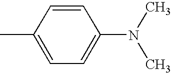 | 1 | 1 |
| A-2 | ″ | ″ | 2 | 2 |
| A-3 | ″ | ″ | 3 | 3 |
| A-4 | 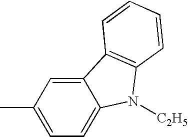 | 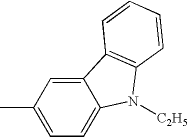 | 2 | 2 |
| A-5 | 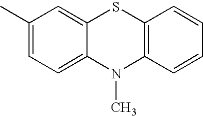 | 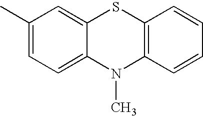 | 3 | 3 |
| A-6 | 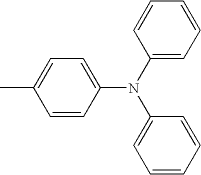 | 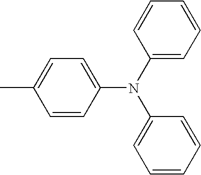 | 2 | 2 |
| A-7 | 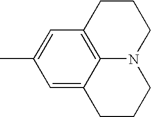 | 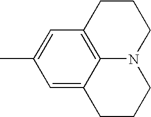 | 1 | 1 |
| A-8 | ″ | ″ | 2 | 2 |

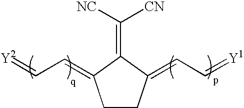

| | Y¹ | Y² | p | q |
|---|---|---|---|---|
| B-1 | 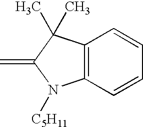 | 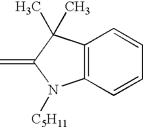 | 1 | 1 |
| B-2 | ″ | ″ | 2 | 2 |
| B-3 | ″ | ″ | 3 | 3 |
| B-4 | 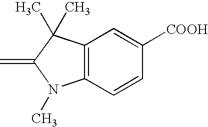 | 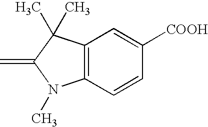 | 2 | 2 |
| B-5 | ″ | ″ | 3 | 3 |
| B-6 | 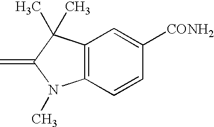 | 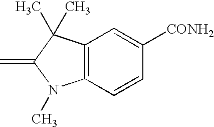 | 2 | 2 |
| B-7 | ″ | ″ | 3 | 3 |
| B-8 | 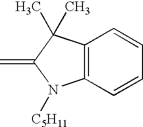 | 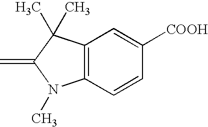 | 2 | 2 |
| B-9 | 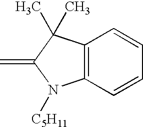 | 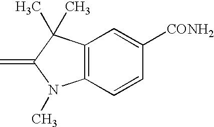 | 2 | 2 |
| B-10 | 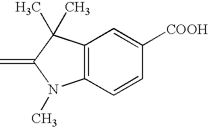 | 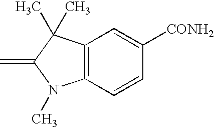 | 2 | 2 |
| B-11 | 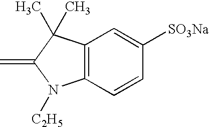 | 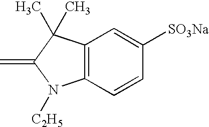 | 2 | 2 |
| B-12 | 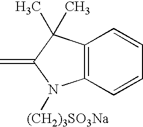 | 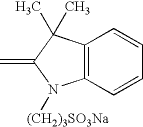 | 2 | 2 |
| B-13 | 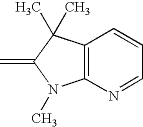 | 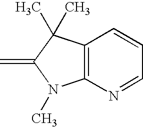 | 2 | 2 |
| B-14 | 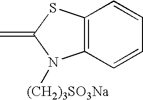 | 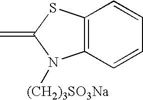 | 1 | 1 |
| B-15 | 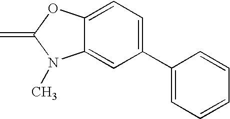 | 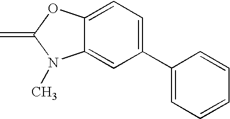 | 1 | 1 |
| B-16 | 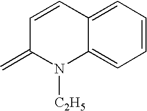 | 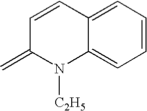 | 1 | 1 |
| B-17 | 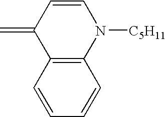 | 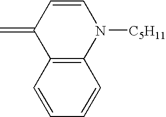 | 1 | 1 |
| B-18 | 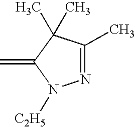 | 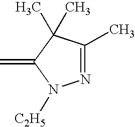 | 2 | 2 |
| B-19 | 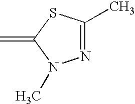 | 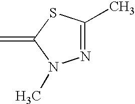 | 2 | 2 |

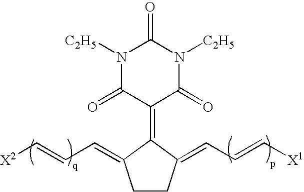

| | X¹ | X² | p | q |
|---|---|---|---|---|
| C-1 | 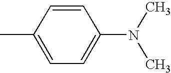 | 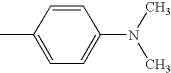 | 1 | 1 |
| C-2 | ″ | ″ | 2 | 2 |
| C-3 | ″ | ″ | 3 | 3 |
| C-4 | 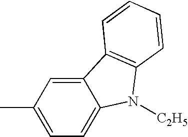 | 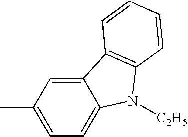 | 2 | 2 |
| C-5 | 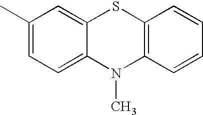 | 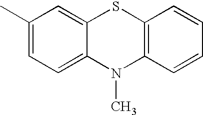 | 3 | 3 |
| C-6 | 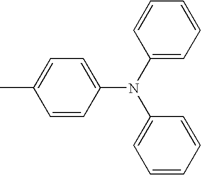 | 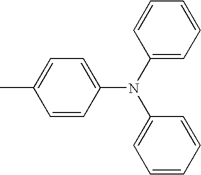 | 2 | 2 |
| C-7 | 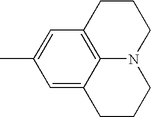 | 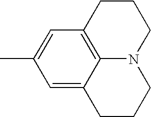 | 1 | 1 |
| C-8 | ″ | ″ | 2 | 2 |

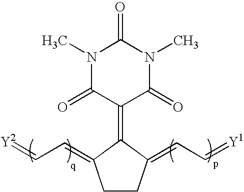

| | Y¹ | Y² | p | q |
|---|---|---|---|---|
| AD-1 | 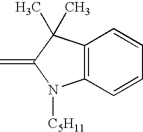 | 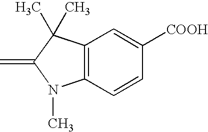 | 3 | 3 |
| AD-2 | 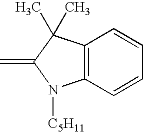 | 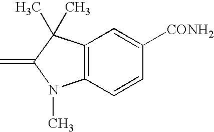 | 2 | 2 |
| AD-3 | 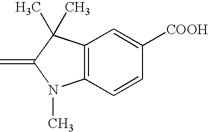 | 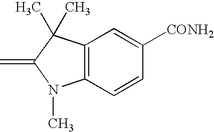 | 3 | 3 |
| AD-4 | 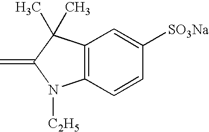 | 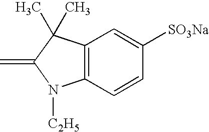 | 2 | 2 |
| AD-5 | 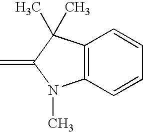 | 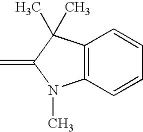 | 3 | 3 |

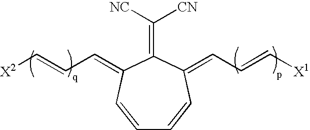

| | X¹ | X² | p | q |
|---|---|---|---|---|
| E-1 | 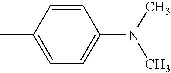 | 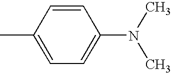 | 1 | 1 |
| E-2 | ″ | ″ | 2 | 2 |
| E-3 | ″ | ″ | 3 | 3 |
| E-4 | 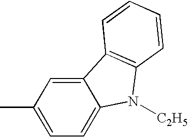 | 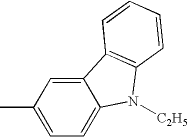 | 2 | 2 |
| E-5 | 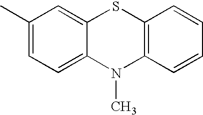 | 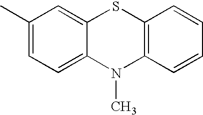 | 3 | 3 |
| E-6 | 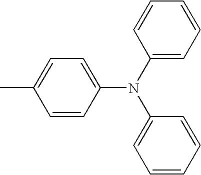 | 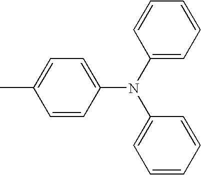 | 2 | 2 |
| E-7 | 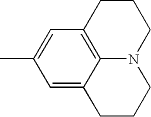 | 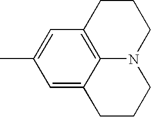 | 1 | 1 |
| E-8 | ″ | ″ | 2 | 2 |

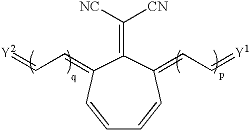

| | Y¹ | Y² | p | q |
|---|---|---|---|---|
| F-1 | 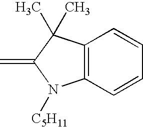 | 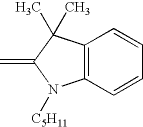 | 3 | 3 |
| F-2 | 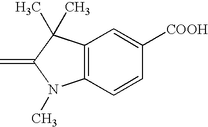 | 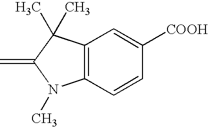 | 3 | 3 |
| F-3 | 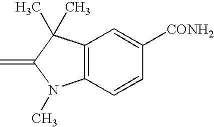 | 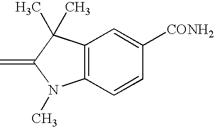 | 3 | 3 |
| F-4 | 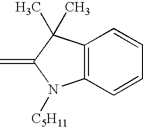 | 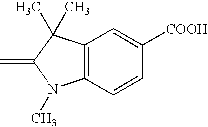 | 3 | 3 |
| F-5 | 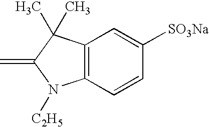 | 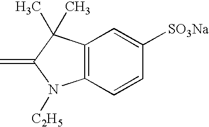 | 2 | 2 |
| F-6 | 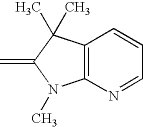 | 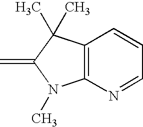 | 3 | 3 |

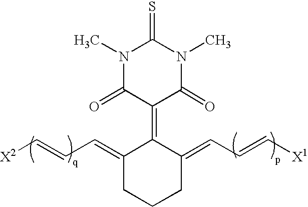

| | X¹ | X² | p | q |
|---|---|---|---|---|
| G-1 | 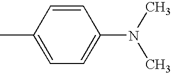 | 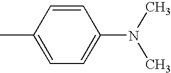 | 1 | 1 |
| G-2 | ″ | ″ | 2 | 2 |
| G-3 | ″ | ″ | 3 | 3 |
| G-4 | 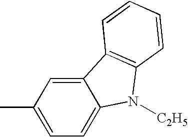 | 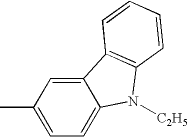 | 2 | 2 |
| G-5 | 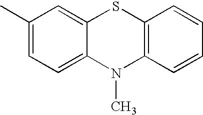 | 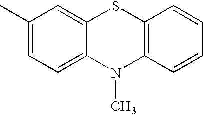 | 3 | 3 |
| G-6 | 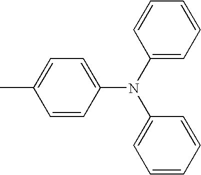 | 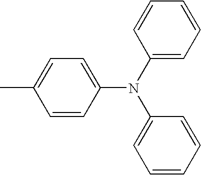 | 2 | 2 |
| G-7 | 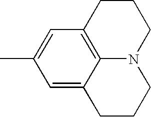 | 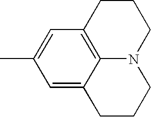 | 1 | 1 |
| G-8 | ″ | ″ | 2 | 2 |

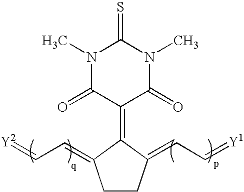

| | Y¹ | Y² | p | q |
|---|---|---|---|---|
| H-1 | 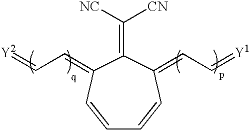 | 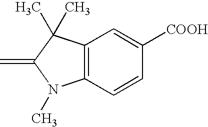 | 3 | 3 |
| H-2 | 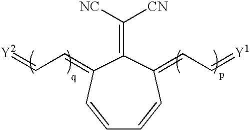 | 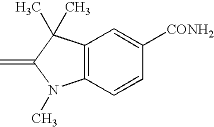 | 2 | 2 |
| H-3 | 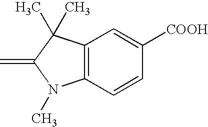 | 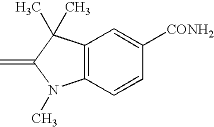 | 3 | 3 |
| H-4 | 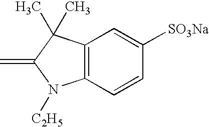 | 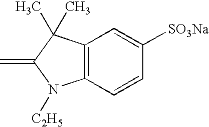 | 2 | 2 |
| H-5 | 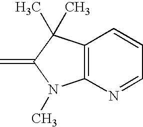 | 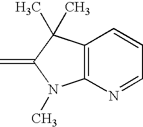 | 3 | 3 |

It is preferred that the compound according to the present invention is a compound having a two-photon absorbing cross sectional area (δ: delta) of 1,000 GM (1 GM=1×10⁻⁵⁰ cm⁴·s/photon) or more for the improvement of sensitivity and a recording velocity when it is used as a two-photon absorbing material, and for the miniaturization of laser beams at time of recording, more preferably 3,000 GM or more, and still more preferably 5,000 GM or more. The value of a two-photon absorbing cross sectional area used is a value evaluated according to the measuring method shown in the following example.

Further, it is preferred to irradiate the compound of the present invention with laser beams having wavelengths longer than the linear absorption band of the compound to induce non-resonant two-photon absorption, it is preferred to irradiate the compound with laser beams of wavelengths longer than the linear absorption band of the compound and from 400 to 1,600 nm to induce non-resonant two-photon absorption from the point of the recording density in using in two-photon absorbing (three dimensional) optical recording material, and it is more preferred to induce non-resonant two-photon absorption by irradiating with laser beams of from 400 to 1,000 nm.

It is more preferred to irradiate the compound of the present invention with laser beams having wavelengths longer than the linear absorption band of the compound, to thereby induce non-resonant two-photon absorption, and to generate emission from the above excitation state.

The compound according to the present invention may induce three-photon or higher non-resonant multi-photon absorption and non-resonant multi-photon emission.

The content of the dye in the two-photon absorbing material is not particularly limited, but may be properly selected from the range of 0.001 to 100% by weight based on the use thereof.

The two-photon absorbing material of the present invention can be applied to an optical recording medium, a three dimensional optical recording medium, a three dimensional volume display, three dimensional stereolithography, two-photon shadow-making, two-photon photo-dynamic therapy (PDT) and up-conversion laser.

### EXAMPLE

The specific examples of the present invention are described below on the basis of the experiment results.

### Example 1

### Synthesis of Compound D-1

Exemplified Compound D-1 can be synthesized according to the following method. Other compounds according to the present invention can also be synthesized by the synthesis method of Compound D-1 and the methods described in Tetrahedron Lett., 42, 6129 (2001). However, the synthesis methods of the compounds of the present invention are not limited thereto.

### Synthesis Example of Compound D-1

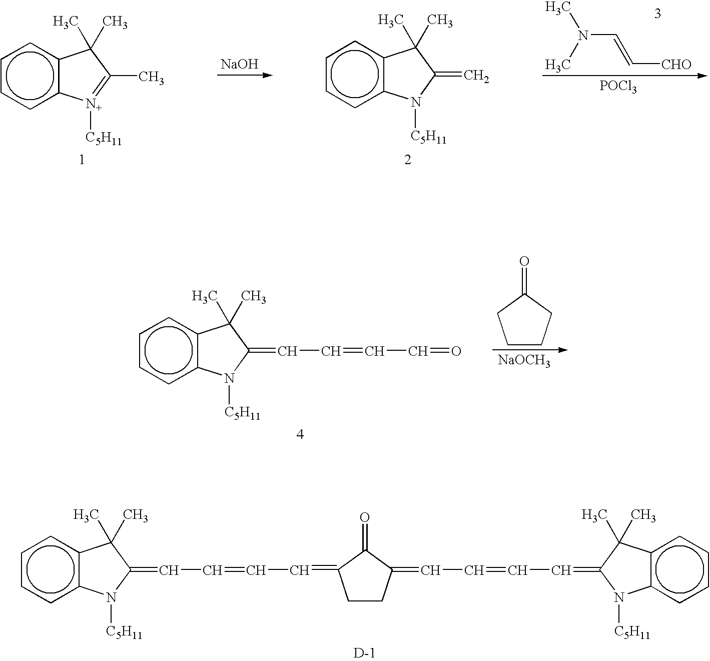

Quaternary salt 1 (14.3 g) (40 mmol) was dissolved in 50 ml of water, 1.6 g (40 mmol) of sodium hydroxide was added thereto and the solution was stirred for 30 minutes at room temperature. The reaction mixture was subjected to extraction three times with ethyl acetate, and concentrated after being dried over magnesium sulfate, thereby 9.2 g (yield: 100%) of oil of methylene base 2 was obtained.

Dimethylaminoacrolein 3 (3.97 g) (40 mmol) was dissolved in 50 ml of acetonitrile, 6.75 g (44 mmol) of phosphorus oxychloride was dropwise added thereto with cooling at 0° C., and the reaction solution was stirred at 0° C. for 10 minutes. Subsequently, an acetonitrile solution containing 9.2 g of methylene base 2 was dropwise added to the above solution, and the solution was stirred at 35° C. for 4 hours. After pouring the solution to 100 ml of ice water, 16 g of sodium hydroxide was added, and the solution was refluxed for 10 minutes. After cooling, the reaction solution was subjected to extraction three times with ethyl acetate, drying over magnesium sulfate, and then concentration. The concentrate was refined with silica gel column chromatography (developing solvents: ethyl acetate/hexane=1/10 to 1/3), thereby 4.4 g (yield: 39%) of oil of aldehyde 4 was obtained.

Cyclopentanone (0.126 g) (1.5 mmol) and 0.85 g (3 mmol) of aldehyde 4 were dissolved in 30 ml of dehydrated methanol, and the solution was refluxed under nitrogen atmosphere in the dark. After the reaction solution was homogenized, 0.69 g (3.6 mmol) of a methanol solution containing 28% of sodium methoxide was added thereto, followed by refluxing for further 6 hours. The precipitated crystal after cooling was filtered out and washed with methanol, thus 0.50 g (yield: 54%) of a dark green crystal of Compound D-1 was obtained. The structure of Compound D-1 was confirmed by NMR spectrum, MS spectrum and elementary analysis.

### Synthesis of Compound DD-2

Compound DD-2 of the present invention can be synthesized according to the following method.

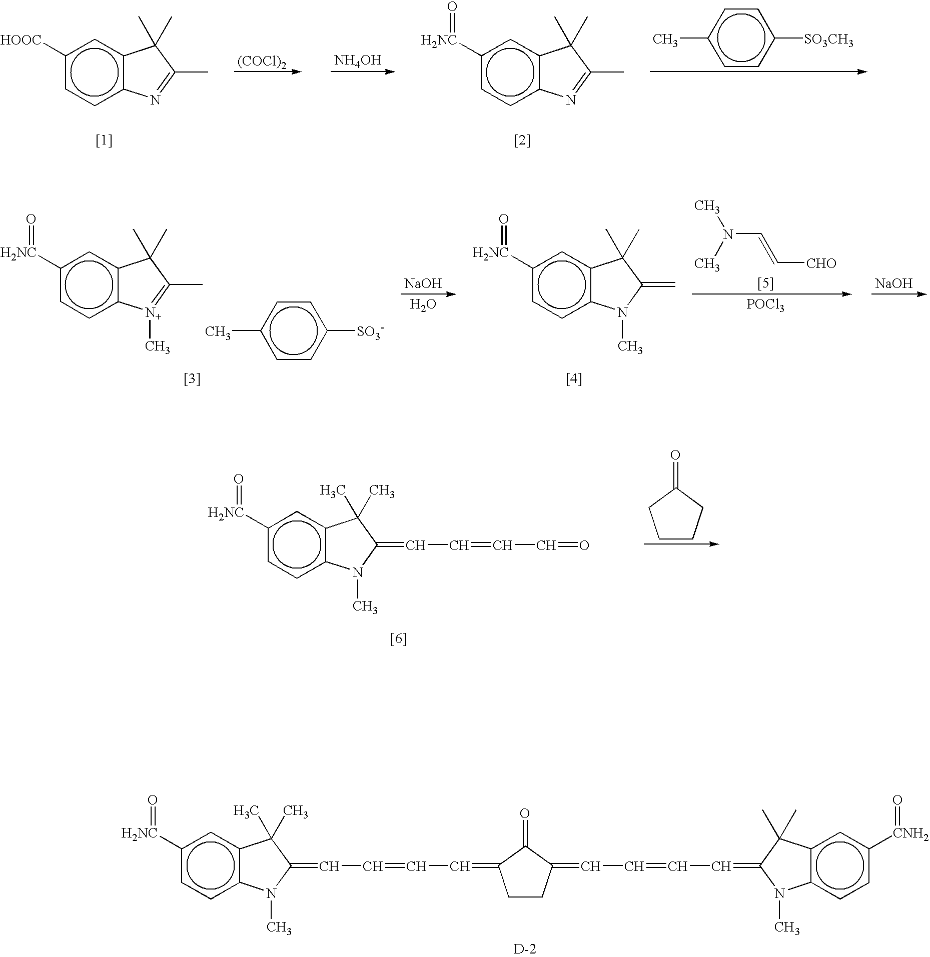

Carboxylic acid [1] (12.2 g) (60 mmol), 0.1 g of DMF and 100 ml of methylene chloride were stirred at room temperature, 11.4 g (90 mmol) of oxalyl chloride was dropwise added thereto, and the reaction mixture was stirred for further 2 hours. After concentration, 80 ml of THF was added and the mixture was stirred, and then the mixture was dropwise added to 36.4 g (0.6 mol) of a 28% aqueous ammonia and 80 ml of a THF solution at 0° C. The temperature of the solution was raised to room temperature with stirring. The reaction solution was subjected to extraction three times with ethyl acetate, drying over magnesium sulfate, and then concentration. The concentrate was refined with silica gel column chromatography (developing solvents: ethyl acetate/methanol=20/1), thereby 4.6 g (yield: 38%) of carbonamide [2] was obtained.

Carbonamide [2] (4.5 g) (22.3 mmol) and 4.97 g (26.7 mmol) of methyl p-toluenesulfonate were heated at 150° C. with stirring. After the mixture was solidified, anisole was added thereto and stirred for 2 hours. The mixture was cooled, and then ethyl acetate was added. The resulted crystal was filtered out and washed with ethyl acetate, thereby the crystal of quaternary salt [3] was obtained.

Quaternary salt [3] was dissolved in 30 ml of water, 1 g (25 mmol) of sodium hydroxide was added to the solution, followed by stirring at room temperature for 30 minutes. The reaction solution was subjected to extraction three times with ethyl acetate, drying over magnesium sulfate, and then concentration, thereby 3.6 g (yield: 75%) of oil of methylene base [4] was obtained.

Dimethylaminoacrolein [5] (3.57 g) (36 mmol) was dissolved in 50 ml of acetonitrile, 4.60 g (30 mmol) of phosphorus oxychloride was dropwise added thereto with cooling at 0° C., and the reaction solution was stirred at 0° C. for 10 minutes. Subsequently, an acetonitrile solution containing 3.6 g (16.6 mmol) of methylene base [4] was dropwise added to the above solution, and the solution was stirred at 40° C. for 4 hours. After pouring the solution to 100 ml of ice water, 6 g of sodium hydroxide was added, and the solution was refluxed for 10 minutes. After cooling, the reaction solution was subjected to extraction three times with ethyl acetate, drying over magnesium sulfate, and then concentration. The concentrate was refined with silica gel column chromatography (developing solvents: ethyl acetate/methanol=20/1), thereby 1.64 g (yield: 35%) of aldehyde [6] was obtained.

Cyclopentanone (0.134 g) (1.6 mmol) and 0.86 g (3.2 mmol) of aldehyde [6] were dissolved in 60 ml of dehydrated methanol, 0.78 g (4 mmol) of a methanol solution containing 28% of sodium methoxide was added thereto, and the solution was refluxed under nitrogen atmosphere in the dark for 4 hours. After concentration, the concentrate was refined with silica gel column chromatography (developing solvents: ethyl acetate/methanol=5/1), dispersed in ethyl acetate-hexane and filtered out, thereby 0.42 g (yield: 45%) of a dark green crystal of objective DD-2 was obtained. The structure of DD-2 was confirmed by NMR spectrum, MS spectrum and elementary analysis.

### Synthesis of Compound A-1

Compound A-1 of the present invention was synthesized according to the following method. However, the synthesis of the compound is not limited thereto.

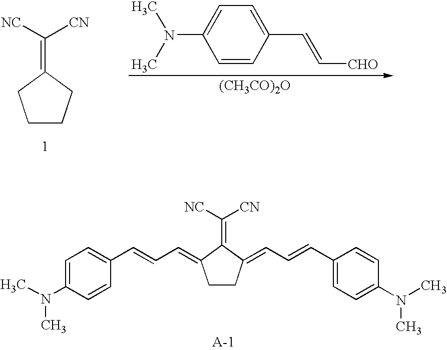

Cyclopentylidene malononitrile (1), the starting compound in the above reaction scheme, was synthesized according to the method described in Julian Mirek et al., Synthesis, Vol. 4, p. 296 (1980).

Starting compound (1) (1.3 g) (10 mmol) and 3.5 g (20 mmol) of N,N-dimethylaminocinnamaldehyde were suspended in 30 ml of acetic anhydride, stirred at 130° C. for 2 hours, and then refluxed for 2 hours. After the reaction solution was allowed to stand for cooling, 100 ml of ethyl acetate was added thereto, and the crystal precipitated was filtered out. The obtained crude crystal was suspended in 100 ml of ethyl acetate, washed for 1 hour with refluxing, and then filtered out, thereby 0.32 g (yield: 7.1%) of golden crystal of Compound A-1 was obtained.

¹H NMR spectrum (solvent: DMSO-d⁶): 2.82 (s, 4H), 3.01 (s, 12H), 6.72 (d, 4H), 6.99 (m, 4H), 7.55 (d, 4H), 7.80 (d, 2H)

### Synthesis of Compound B-2

Compound B-2 of the present invention was synthesized according to the following method. However, the synthesis of the compound is not limited thereto.

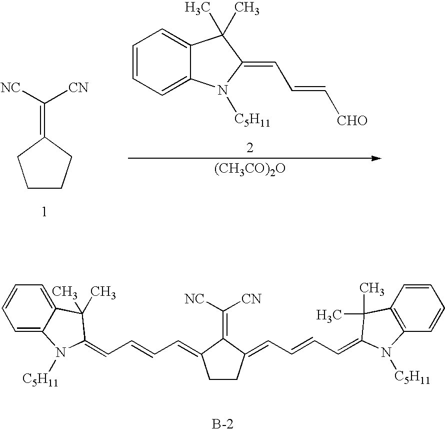

Starting compound (1) (0.65 g) (5 mmol) and 2.8 g (10 mmol) of aldehyde compound (2) in the above reaction scheme were dissolved in 20 ml of acetic anhydride. After the solution was stirred in the dark under argon gas flow at room temperature for 7 hours, distilled water was added. The solution was then subjected to extraction with ethyl acetate. The crude product obtained was refined with silica gel column chromatography (eluate: ethyl acetate/hexane=1/3), thereby 0.6 g (yield: 18%) of a dark blue oil of objective Compound B-2 was obtained.

¹H NMR spectrum (solvent: CDCl₃-d¹): 0.93 (m, 8H), 1.38 (m, 6H), 1.60 (s, 12H), 1.70 (m, 4H), 2.69 (m, 4H), 3.68 (t, 4H), 5.62 (d, 2H), 6.13 (t, 2H), 6.76 (d, 2H), 6.99 (t, 2H), 7.20 (m, 4H), 7.37 (t, 2H), 8.00 (d, 2H)

### Example 2

Evaluating Method of Two-Photon Absorbing Cross Sectional Area:

The evaluation of the two-photon absorbing cross sectional areas of the compounds of the present invention was performed by referring to the method described in M. A. Albota et al., Appl. Opt., 37, 7352 (1998). As the light source of the measurement of the two-photon absorbing cross sectional area, Ti:sapphire pulse laser (pulse width: 100 fs, repetition: 80 MHz, average output: 1 W, peak power: 100 kW) was used, and the two-photon absorbing cross sectional area was measured in the wavelength region of from 700 to 1,000 nm. Rhodamine B and fluorescein were measured as standard substances. The value of the two-photon absorbing cross sectional area of each compound was obtained by compensating the measured value by using the values of the two-photon absorbing cross sectional areas of rhodamine B and fluorescein described in C. Xu et al., J. Opt. Soc. Am. B, 13, 461 (1996).

The two-photon absorbing cross sectional areas of the compounds of the present invention were measured according to the above method, and the obtained values were shown in Table 1 below in GM unit (1 GM=1×10⁻⁵⁰ cm⁴·s/photon). Each value shown in Table 1 was the maximum value of the two-photon absorbing cross sectional areas in the wavelength region measured.

The two-photon absorbing cross sectional areas of Comparative Compounds 1 and 2 each having the structure shown below were measured according to the above method. The results obtained are shown in Table 1.

Comparative Compound 1

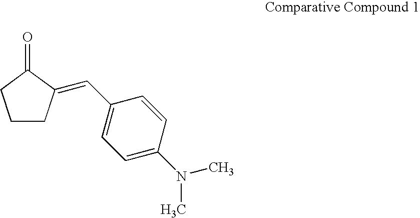

Comparative Compound 2

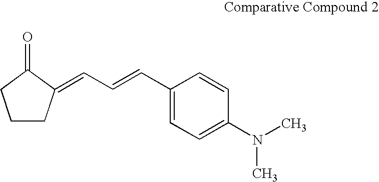

TABLE 1

| | Two-photon | |
|---|---|---|
| | absorbing | |
| | Cross | |
| Compound No. | Section/GM | Solvent and Concentration |
| D-1 | 5,430 | Chloroform (10⁻³ M) |
| D-2 | 3,050 | Chloroform (10⁻³ M) |
| D-3 | 1,510 | Chloroform (10⁻³ M) |
| D-5 | 7,900 | DMSO (10⁻⁴ M) |
| D-8 | 5,550 | Chloroform (10⁻³ M) |
| D-10 | 2,150 | Chloroform (10⁻³ M) |
| D-17 | 1,330 | Chloroform (10⁻³ M) |
| D-26 | 3,450 | Chloroform + 1% triethylamine |
| | | (10⁻³ M) |
| D-31 | 4,000 | Chloroform (10⁻³ M) |
| D-34 | 3,470 | Chloroform (10⁻³ M) |
| D-35 | 10,100 | DMSO (10⁻⁴ M) |
| D-36 | 3,560 | Methanol + 1% triethylamine |
| | | (10⁻⁴ M) |
| D-37 | 6,950 | Methanol + 1% triethylamine |
| | | (10⁻⁴ M) |
| DD-2 | 10,300 | Chloroform (10⁻⁴ M) |
| DD-35 | 8,750 | Methanol + 1% triethylamine |
| | | (10⁻⁴ M) |
| Comparative | 60 | Chloroform (10⁻⁴ M) |
| Compound 1 | | |
| Comparative | 145 | Chloroform (10⁻⁴ M) |
| Compound 2 | | |

As can be seen from the results in Table 1, good characteristics superior to those in the conventional materials can be obtained according to the present invention.

### Example 3

Evaluating Method of Luminous Intensity of Two-Photon Emission

A compound of the present invention was dissolved in chloroform, the emission spectrum of the compound obtained by irradiation with laser pulse of 1,064 nm of Nd:YAG laser was measured, and the luminous intensity of the non-resonant two-photon emission was found from the area of the obtained emission spectrum.

Samples 1 and 2: Each of Compounds D-1 and DD-2 according to the present invention was dissolved in chloroform, and the solutions respectively having concentration of 1×10⁻⁴ M were prepared.

Comparative Compound 1: As a compound emitting strong two-photon emission, the compound disclosed in WO 97/09043 (having the structure shown below) was dissolved in acetonitrile and a solution having concentration of 1×10⁻⁴ M was prepared.

Comparative Compound

(“Dye 1” Disclosed in WO 97/09043)

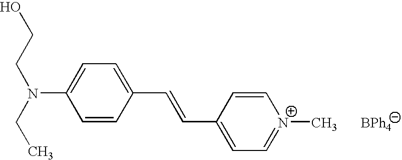

Each of Samples 1 and 2 and Comparative Sample 1 was irradiated with laser pulse of 1,064 nm of Nd:YAG laser on the same condition, and the non-resonant two-photon emission spectrum was measured. The area of the obtained emission spectrum (luminous intensity of non-resonant two-photon emission) of each sample was shown in Table 2 below in a relative value with the value of Comparative Sample 1 as 1.

TABLE 2

| | | Luminous Intensity |
|---|---|---|
| | | of Non-resonant |
| | | Two-photon |
| Sample No. | Compound | emission |
| Sample 1 | D-1 | 24 |
| Sample 2 | DD-2 | 45 |
| Comparative | Dye 1 disclosed | 1 |
| Sample 1 | in WO 97/09043 | |

As can be seen from the results in Table 2, good characteristics superior to that in the conventional material can be obtained according to the present invention.

### Example 4

Evaluating Method of Two-Photon Absorbing Cross Sectional Area:

The evaluation of the two-photon absorbing cross sectional areas was performed according to the Z-scanning method described in Mansoor Sheik-Bahae et al., IEEE. Journal of Quantum Electronics, 26, 760 (1990). The Z-scanning method is a method widely utilized as a measuring method of a nonlinear optical constant, which comprises moving a sample to be measured along laser beams beside the focal point of converged laser beams and recording the variation of the quantity of transmitted light. Since the power density of incident light varies according to the position of a sample, the quantity of transmitted light attenuates beside the focal point in the case where there is nonlinear absorption. The two-photon absorbing cross sectional area was computed by fitting the variation of the quantity of transmitted light to the theoretical curve estimated from the incident light intensity, the converging spot size, the thickness and concentration of the sample. As the light source for measuring the two-photon absorbing cross sectional area, Ti:sapphire pulse laser (pulse width: 100 fs, repetition: 80 MHz, average output: 1 W, peak power: 100 kW) was used, and the two-photon absorbing cross sectional area was measured in the wavelength region of from 700 to 1,000 nm. As the sample for measuring two-photon absorption, a solution obtained by dissolving each compound in chloroform in concentration of 1×10⁻³ M was used.

The two-photon absorbing cross sectional areas of the compounds of the present invention were measured according to the above method, and the obtained values were shown in Table 3 below in GM unit (1 GM=1×10⁻⁵⁰ cm⁴·s/photon).

The two-photon absorbing cross sectional area of the comparative compound having the structure shown below was measured according to the above method. The result obtained is also shown in Table 3.

Comparative Compound

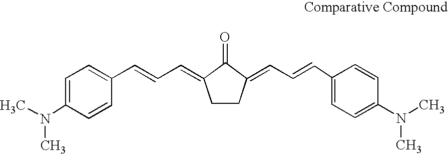

TABLE 3

| | Two-photon | |
|---|---|---|
| | absorbing Cross | |
| | Sectional Area | |
| Compound No. | (GM) | Remarks |
| A-1 | 3,000 | Invention |
| B-2 | 12,000 | Invention |
| Comparative Compound | 950 | Comparative |
| | | Example |

As can be seen from the results in Table 3, good characteristics superior to that in the conventional material can be obtained according to the present invention.

By using the compound according to the present invention, a non-resonant two-photon absorbing and emitting material exhibiting far stronger non-resonant two-photon absorption and two-photon emission than conventional materials can be obtained.

The entire disclosure of each and every foreign patent application: Japanese Patent Applications No. 2002-293809, No. 2002-12417 and No. 2002-70303, from which the benefit of foreign priority has been claimed in the present application is incorporated herein by reference, as if fully set forth.

What is claimed is:

1. A non-resonant two-photon absorbing material comprising a compound represented by the following formula (1) or (3): 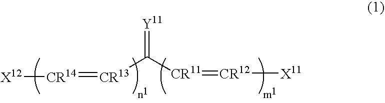
wherein R¹¹, R¹², R¹³ and R¹⁴ each independently represents a hydrogen atom or a substituent, and some of R¹¹, R¹², R¹³ and R¹⁴ may be bonded to each other to form a ring; m¹ and n¹ each represents an integer of from 0 to 4, and when m¹ and n¹ each represents 2 or more, a plurality of R¹¹, R¹², R¹³ and R¹⁴ may be the same or different; X¹¹ and X¹², which may be the same or different, each represents a substituted or unsubstituted aryl group, or a substituted or unsubstituted heterocyclic group; Y¹¹ represents an atomic group containing at least one of partial structures represented by the following formulae (2-1) to (2-6): 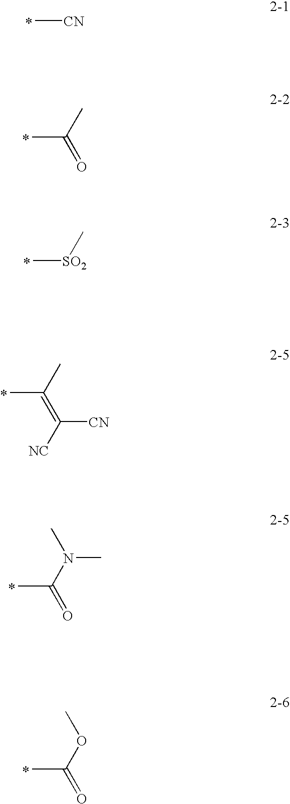
wherein * represents the bonding position in Y¹¹, when Y¹¹ contains two of partial structures represented by formulae (2-1) to (2-6), the two partial structures may be different from each other, and may be bonded to form a ring, and when Y¹¹ contains one of partial structure represented by formulae (2-1) to (2-6), other necessary atom or atomic group may be arbitrary; 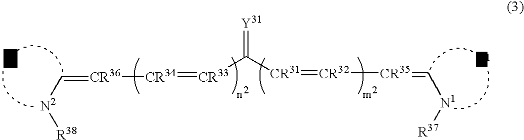
wherein R³¹, R³², R³³, R³⁴, R³⁵ and R³⁶ each independently represents a hydrogen atom or a substituent, and some of R³¹, R³², R³³, R³⁴, R³⁵ and R³⁶ may be bonded to each other to form a ring; m² and n² each represents an integer of from 1 to 4, and when m² and n² each represents 2 or more, a plurality of R³¹, R³², R³³ and R³⁴ may be the same or different; R³⁷ and R³⁸ each represents a hydrogen atom, an alkyl group, an alkenyl group, an aryl group, or a heterocyclic group; Z¹ and Z² each represents an atomic group to form a 5- or 6-membered ring; Y³¹ represents an oxygen atom, or an atomic group containing at least one of partial structures represented by the following formulas (2-1) to (2-6): 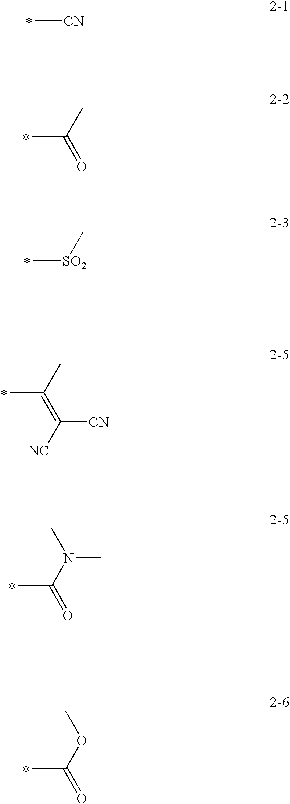
wherein * represents the bonding position in Y³¹ when Y³¹ contains two of partial structures represented by formulae (2-1) to (2-6), the two partial structures may be different from each other, and may be bonded to form a ring, and when Y³¹ contains one of partial structure represented by formulae (2-1) to (2-6), other necessary atom or atomic group may be arbitrary.

2. The non-resonant two-photon absorbing material as claimed in claim 1, wherein Y¹¹ contains two partial structures represented by the formula (2-1).

3. The non-resonant two-photon absorbing material as claimed in claim 1, wherein Y³¹ contains two partial structures represented by the formula (2-1).

4. The non-resonant two-photon absorbing material as claimed in claim 1, wherein Y³¹ represents an oxygen atom.

5. The non-resonant two-photon absorbing material as claimed in claim 1, wherein the ring formed by Z¹ and Z² is an indolenine ring or an azaindolenine ring.

6. The non-resonant two-photon absorbing material as claimed in claim 1, wherein R¹¹ and R¹³, or R³¹ and R³³ are bonded to each other to form a ring.

7. The non-resonant two-photon absorbing material as claimed in claim 1, wherein X¹¹ and X¹² each represents a phenyl group, a naphthyl group or a nitrogen-containing heterocyclic group.

8. The non-resonant two-photon absorbing material as claimed in claim 1, wherein the compound represented by the formula (1) or (3) has a hydrogen bonding group in the molecule.

9. The non-resonant two-photon absorbing material as claimed in claim 8, wherein the hydrogen bonding group is selected from —COOH, —CONHR¹, —SO₃H, —SO₂NHR², —P(O)(OH)OR³, —OH, —SH, —NHR⁴, —NHCOR⁵ and —NR⁶C(O)NHR⁷, in which R¹ and R² each independently represents a hydrogen atom, an alkyl group, an alkenyl group, an aryl group, a heterocyclic group, —COR⁸ or —SO₂R⁹; R³ to R⁹ each indenpendently represents a hydrogen atom, an alkyl group, an alkenyl group, an aryl group or a heterocyclic group.

10. The non-resonant two-photon absorbing material as claimed in claim 1, wherein the compound represented by formula (1) or (3) has a two-photon absorbing cross sectional area of 1,000 GM or more.

11. A non-resonant two-photon emitting material comprising the compound represented by formula (1) or (3) as described in claim 1.

12. A method for inducing a non-resonant two-photon absorption, which comprises irradiating the compound represented by formula (1) or (3) as described in claim 1 with laser beams having a wavelength longer than the linear absorption band of the compound to induce a two-photon absorption.

13. A method for generating an emission of non-resonant two-photon, which comprises irradiating the compound represented by formula (1) or (3) as described in claim 1 with laser beams having a wavelength longer than the linear absorption band of the compound to induce non-resonant two-photon absorption and generate an emission.

14. An optical recording medium comprising the non-resonant two-photon absorbing material as claimed in claim 1.

15. A three dimensional optical recording medium comprising the non-resonant two-photon absorbing material as claimed in claim 1.

16. A three dimensional volume display comprising the non-resonant two-photon absorbing material as claimed in claim 1.

17. A three dimensional stereolithography material comprising the non-resonant two-photon absorbing material as claimed in claim 1.
